(19) United States
(12) Patent Application Publication
(10) Pub. No.: US 2014/0005199 A1
(43) Pub. Date: Jan. 2, 2014

(54) IMPLANT FOR THE CONTROLLED RELEASE OF PHARMACEUTICALLY ACTIVE AGENTS

(71) Applicants: Ameena Wadee, Johannesburg (ZA); Viness Pillay, Johannesburg (ZA); Yahya Essop Choonara, Johannesburg (ZA); Lisa Claire Du Toit, Johnnesburg (ZA)

(72) Inventors: Ameena Wadee, Johannesburg (ZA); Viness Pillay, Johannesburg (ZA); Yahya Essop Choonara, Johannesburg (ZA); Lisa Claire Du Toit, Johnnesburg (ZA)

(73) Assignee: University of the Witwatersrand, Johannesburg

(21) Appl. No.: 13/989,346

(22) PCT Filed: Nov. 28, 2011
(86) PCT No.: PCT/IB2011/055343

§ 371 Date: Sep. 10, 2013

(30) Foreign Application Priority Data
Nov. 26, 2010 (ZA) 2010-03745

(51) Int. Cl. A61K 9/00; A61K 45/06; A61K 47/32; A61K 31/519

(52) U.S. Cl. CPC A61K 9/0024; A61K 31/519; A61K 45/06; A61K 47/32
USPC 514/249; 514/772.1

(57) ABSTRACT

A pharmaceutical composition or dosage form is described for the delivery of at least one pharmaceutically active agent or drug in a sustained and controlled manner. The pharmaceutical composition is an injectable formulation which is capable of responding to local stimuli at the site of injection, such as pH and temperature, and comprises a thermoresponsive polymer composition with a suspension of pH responsive micro- or nano-particles which contain the at least one pharmaceutically active agent or drug. The thermoresponsive polymer composition is formed from poly(methyl vinyl ether) (PMVE), and an inorganic salt, and the micro- or nano-particles are formed from chitosan and eudragit. The composition can be used to treat any disease or condition which results in a decrease in pH, such as for treating a solid tumour, gout, acidosis, ketosis and the like.

### FIELD OF THE INVENTION

The present invention relates to a pharmaceutical composition or dosage form formed from a polymer solution which is capable of forming an implant following injection into the body due to its thermoresponsive nature and which contains pH responsive micro- or nano-particles which will respond to the site of injection to release entrapped drugs in a sustained manner.

### BACKGROUND TO THE INVENTION

Traditionally implants have been devices which require surgical insertion and removal and for this reason did not have the benefit of patient compliance, and in addition incurred costs due to the required surgical procedures. The most successful of these implants are the Gliadel™ implants currently available for the treatment of malignant human glioma. However, recently focus has shifted to the development of implant systems which can be injected into the body and which are biodegradable and therefore do not require surgical removal. Some such injectable implant systems employ the use of biodegradable polymers together with an organic solvent and the implant is formed in vivo by precipitation of the polymer due to the diffusion of the organic solvent after injection into the body (Packhauser et al., 2004). A disadvantage of such a system is the possible toxicity of the organic solvents utilised.

Chemotherapy, which uses chemical agents (anticancer drugs) to kill cancer cells, is one of the primary methods of cancer treatment. Unfortunately, these anticancer drugs have limited selectivity for cancer and are inherently toxic to both cancer and normal tissues. As a result, anticancer drugs can cause severe side effects and damage to healthy tissues. For example cisplatin is a well-known metal complex that exhibits high antitumor. However, it has significant toxicity, in particular, acute as well as chronic nephrotoxicity. Other common side effects of anticancer drugs include decrease in the number of white blood cells (increasing risk of infection), red blood cells (losing energy) and platelets (risk for bruising and bleeding) as well as nausea, vomiting, hair loss and the like. Furthermore, the high glomerular clearance of the anticancer drugs leads to an extremely short circulation period in the blood compartment. Treatments in conventional dosage form of these drugs may lead to initial cancer regression, but the cancer may also become insensitive to the drugs, causing cancer progression and death.

Of the various approaches developed for targeted drug delivery, polymer nanoparticle technique has been attracting increasing attention since it offers suitable means to deliver drugs to tissues or cells. However, the prior art has several drawbacks. The premature burst release of drugs in bloodstream is a general problem of existing nanoparticle drug carriers, as only a portion of drugs reach the tumors, causing non-targeted drug release, low drug efficiency, toxicity to healthy tissues and less drug being available to cancer. Nanoparticles also have a slow drug release. After the initial burst release, the drug release from nanoparticles becomes very slow. Cancer cells have many forms of over-expressed drug resistance. If the drug influx into the cancer cell is too low, the drug cannot build up a concentration higher than the cell-killing threshold concentration for effective killing. Yet a further issue of nanoparticles is their slow cellular uptake by cancer cells.

### SUMMARY OF THE INVENTION

According to a first embodiment of the invention, there is provided a pharmaceutical composition for the delivery of a pharmaceutically active agent, the composition comprising:
a thermoresponsive polymer composition comprising poly(methyl vinyl ether) (PMVE) and an inorganic salt, wherein the composition is in a liquid form at or about room temperature and in a solid or gelatinous form at or about body temperature; and
a plurality of micro- or nano-particles which are pH responsive and which include at least one pharmaceutically active agent;
wherein the micro- or nano-particles are suspended in the thermoresponsive polymer composition.

The inorganic salt may be calcium chloride or sodium hydrogen phosphate. The thermoresponsive polymer compositon may also comprise a second polymer, such as gum arabic, carageenan, hydroxypropyl cellulose (HPC), methylcellulose (MC) and hydroxypropylmethylcellulose (HPMC).

The micro- or nano-particles may comprise at least two pH responsive polymers, such as chitosan and eudragit. The micro- or nano-particles may also comprise additional polymers, such as alginates and/or HPMC, to stabilise the particles and/or to enhance entrapment of the pharmaceutically active agent.

The pharmaceutically active agent may be a chemotherapeutic agent, for example for treating a solid tumour such as a liver tumour.

The pharmaceutically active agent may be for treating pain.

The pharmaceutically active agent may be released in a sustained and/or controlled manner for a long-term therapeutic effect.

The thermoresponsive polymer composition may also comprise a second pharmaceutically active agent.

The pharmaceutical formulation may be an injectable formulation.

According to a second embodiment of the invention, there is provided a method of introducing a pharmaceutically active agent to a specific site within a human or animal comprising introducing to the specific site in the human or animal a pharmaceutical composition substantially as described above.

According to a third embodiment of the invention, there is provided a method of treating a solid cancerous tumour comprising percutaneously injecting a pharmaceutical composition substantially as described above to the site of the tumour.

According to a fourth embodiment of the invention, there is provided a method of formulating a pharmaceutical composition substantially as described above, the method comprising the steps of:
forming pH responsive micro- or nano-particles from at least two pH responsive polymers and at least one pharmaceutically active agent;
forming a thermoresponsive polymer composition from a thermoresponsive polymer, PMVE, and an inorganic salt; and
mixing the micro- or nano-particles with the thermoresponsive polymer composition so that the micro- or nano-particles are suspended in the theremoresponsive polymer composition.

### BRIEF DESCRIPTION OF THE FIGURES

FIG. 1: is a graph showing the typical effect of increasing temperature on the viscoelasticity of thermoresponsive polymer formulations according to the present invention.

FIG. 2: shows the effect of calcium chloride on the gelation temperature of the formulations.

FIG. 3: shows the effect of polymer concentration on the dynamic viscosity of the formulations at 37.5° C.

FIG. 4: shows the release of folic acid from three formulations containing the same amounts of calcium chloride but varying amounts of PMVE.

FIG. 5: shows the release of folic acid from particles of the present invention in different pH values.

FIG. 6: shows electron micrographs of the spindle-like morphology of the particles formed according to the present invention.

FIG. 7: shows the release of implants loaded with methotrexate.

FIG. 8: shows the release of folic acid from implants cooled with ice.

### DETAILED DESCRIPTION OF THE INVENTION

The invention provides a pharmaceutical composition or dosage form for the delivery of at least one pharmaceutically active agent or drug in a sustained and controlled manner. The pharmaceutical composition is typically an injectable formulation which is capable of responding to local stimuli at the site of injection, such as pH and temperature, and comprises a thermoresponsive polymer composition with a suspension of pH responsive micro- or nano-particles which contain the at least one pharmaceutically active agent or drug.

For example, solid tumours are reported to have different environments when compared to normal cells due to the high metabolic activity occurring at the sites of cancers. For this reason, the temperature of a tumour is often higher than surrounding areas (about 37.5-38° C.), the pH of the environment is lower and the environment of a solid tumour also lacks oxygen. The temperature of the tumour can be used as a stimulus for in situ gel formation of the injected composition so as to form an implant if the pharmaceutical composition is thermoresponsive.

However, the invention is not intended to be limited to use in treating tumours, and could be used for treating other diseases or conditions which result in a decrease in pH in the regions in which they occur, such as inflammation, infection, gout, acidosis or ketosis.

As used herein, the term “sustained release” refers to the continual release of a drug or active agent or any combination thereof over a period of time.

As used herein, the term “controlled release” refers to control of the rate and/or quantity of a drug or active agent delivered according to the drug delivery formulations of the invention. The controlled release can be continuous or discontinuous, and/or linear or non-linear. This can be accomplished using one or more types of polymer compositions, drug loadings, excipients or degradation enhancers, or other modifiers, administered alone, in combination or sequentially to produce the desired effect. The rate of release of a drug or active agent from the micro- or nano-particles or from the thermoresponsive polymer composition also depends on the quantity of the loaded drug or active agent as a percent of the final product formulation. Yet another factor that affects the release rate of the drug or active agent from the micro- or nano-particles is the particle size of the drug or active agent. By adjusting these factors, degradation, diffusion, and controlled release may be varied over very wide ranges. For example, release may be designed to occur over hours, days, or months.

As used herein, the term “pH responsive polymer” refers to a polymer which is insoluble at the pH of healthy tissue, but soluble at the pH of cancer cells. Healthy tissue pH as used in this specification means the pH of non-cancerous tissues and is most typically approximately 7.4. The pH of cancerous tissues is in the range of between about 4.5 and 7.2 and most typically is below about 7.0.

As used herein, the term “pharmaceutically active agent” refers to any compound or composition which, when administered to a human or animal induces a desired pharmacologic, immunogenic, and/or physiologic effect by local and/or systemic action. The term therefore encompasses those compounds or chemicals traditionally regarded as drugs, vaccines, and biopharmaceuticals intended for use in the diagnosis, characterization, cure, mitigation, treatment, prevention or allaying the onset of a disease, disorder, or other condition. These include molecules such as proteins, peptides, hormones, nucleic acids, gene constructs and the like. The term “pharmaceutically active agent” includes compounds or compositions for use in all of the major therapeutic areas including, but not limited to, anti-infectives such as antibiotics and antiviral agents; analgesics and analgesic combinations; local and general anesthetics; anorexics; antiarthritics; antiasthmatic agents; anticonvulsants; antidepressants; antihistamines; anti-inflammatory agents; antinauseants;

antimigraine agents; antineoplastics; antipruritics; antipsychotics; antipyretics; antispasmodics; cardiovascular preparations (including calcium channel blockers, β-blockers, β-agonists and antiarrhythmics); antihypertensives; chemotherapeutics; diuretics; vasodilators; central nervous system stimulants; decongestants; diagnostics; hormones; bone growth stimulants and bone resorption inhibitors; immunosuppressants; muscle relaxants; psychostimulants; sedatives;

tranquilizers; proteins, peptides, and fragments thereof; and nucleic acid molecules. Anti-cancer drugs include 6-mercaptopurine, ara-CMP, bleomycin, busulfan, camptothecin sodium salt, carboplatin, carmustine, chlorambucil, chlorodeoxyadenosine, cisplatin, cyclophosphamide, cytarabine, dacarbazin, dactinomycin, daunorubicin, docetaxel, doxorubicin, etoposide, floxuridine, fludarabine phosphate, fluorouracil, gemcitabine, hexamethyl melamine, hydroxyurea, idarubicin, iphosphamide, irinotecan, lomustine, mechlorethamine, melphalan, methotrexate, mithramycin, mitomycin, mitotane, mitoxantrone, navelbine, paclitaxel, pentostatin, pipobroman, procarbazine, streptozocin, teniposide, thioguanine, thiotepa, topotecan, triethylene melamine, trimetrexate, uracil nitrogen mustard, vinblastine, vincristine, and all other anticancer drugs.

The thermoresponsive polymer composition comprises poly(methyl vinyl ether) (PMVE) (a thermoresponsive polymer) and an inorganic salt, such as calcium chloride or sodium hydrogen phosphate. It can also comprise a second polymer such as gum arabic, carageenan, hydroxypropyl cellulose (HPC), methylcellulose (MC), ethylcellulose and hydroxypropylmethylcellulose (HPMC). The thermoresponsive polymer composition is designed to reversibly transition from a solution at about ambient room temperature (about 20° C.) to a solid or semi-solid (gel) by about body temperature (about 37° C.).

PMVE is a water soluble, biocompatible polymer which displays thermoresponsiveness and is reported to have a lower critical solution temperature (LCST) of 32-38° C. (Karayanni and Staikos, 2000; Madbouly and Ougizawa, 2005). It converts from a solution into a gel instantaneously upon heating to its lower critical solution temperature (LCST). The applicant is not aware of any description of the use of PMVE in the formulation of a thermoresponsive system for the treatment of cancer

The micro- or nano-particles comprises at least two pH responsive polymers, and in particular, chitosan and eudragit. Chitosan is an abundant natural polysaccharide obtained from the deacetylation of chitin, a component of the external skeleton of many crustaceans and insects. It is a cationic polysaccharide that has one amino group and two free hydroxyl groups in every monomer unit. The presence of the amino groups gives the molecule an overall positive charge.

Poly(methacrylic acid-co-methyl methacrylate)—commercially available as Eudragit S100 and Eudragit L100—has both carboxyl groups and ester groups. Eudragit S100, the polymer utilised in this study, has a ratio of carboxyl groups to ester groups of 1:2. The polymer hence carries an overall negative charge. The combinations of these two polymers give rise to an interpolymeric complex based on the interaction of these two charged polymers.

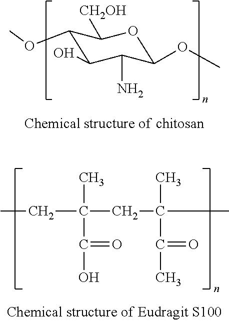

The interaction that takes place between these two polymers can be used to formulate micro- or nano-particles according to the invention. The micro- or nano-particles can also comprise additional polymers, such as alginates and/or HPMC, to stabilise the particles and/or to enhance entrapment of the pharmaceutically active agent. The microparticles typically comprise from about 5 to about 60% w/v of the pharmaceutical composition.

In one embodiment, the pharmaceutically active agent is a chemotherapeutic agent, for example for treating a solid tumour such as a liver tumour. Suitable chemotherapeutic agents for use in the invention include, but are not limited to, alkylating agents, antimetabolites such as methotrexate, antibiotics, natural or plant derived products, hormones and steroids (including synthetic analogues), and platinum drugs such as cisplatin or carboplatin. Methotrexate was one of the model drugs used herein.

In another embodiment, the pharmaceutically active agent can be for treating pain.

The pharmaceutical composition can also contain a second pharmaceutically active agent for either a long term or a short term therapeutic effect or treatment.

The pharmaceutical composition can be for local or systemic delivery of the active agent or drug, but is particularly suitable for targeted delivery at or near the site of injection.

The pharmaceutically active agent or drug can be loaded into or onto the micro- or nano-particles which are suspended within the thermoresponsive polymer composition. Alternatively or in addition, the active agent or drug can be suspended in the thermoresponsive polymer composition or dissolved within it. The drug or active agent can be added to the polymers used to make the thermoresponsive polymer composition prior to, during, or after the dissolution of the polymers in solution. Preferably, the drug or active agent is added prior to the dissolution of the polymer in solution to facilitate a more uniform dispersion or dissolution of the drug or active agent.

The pharmaceutical composition of the invention provides optimal delivery of a drug or therapeutic agent, as it releases the drug or therapeutic agent in a controlled manner over a desired period of time, such as for at least one month. A slower and steadier rate of delivery may in turn result in a reduction in the frequency with which the drug or therapeutic agent must be administered.

In one embodiment, the implant which is formed in the body can be cooled to cause the implant to transition back to a liquid state. For example, an ice pack may be applied to the skin in the region of the implant. The liquid composition will allow the active agent or drug to be released more quickly, which could be particularly suitable if the active agent is, for example, for pain relief.

The use of implants formed by pharmaceutical compositions according to the present invention for the treatment of solid tumours has several benefits. Firstly, a significantly higher dose of the chemotherapeutic can be administered. High doses of cytotoxic drugs by systemic delivery are limited by toxicity to healthy body cells. With an implant at the site of the tumour this is overcome. Secondly, the chances of systemic side-effects are much reduced, again due to the localized therapy exerted by the implant. Thirdly, the implant can be formulated to release the drug over a number of weeks, improving patient acceptability as it decreases the need of the patient to return to the hospital for systemic treatment or removes the need to take medication daily. Fourthly, as the pharmaceutical composition can be injected into the body and is biodegradable, it is minimally invasive and removes the need for surgical implantation and subsequent surgical removal.

The use of statistical experimental designs to develop pharmaceutical drug delivery systems offers a more efficient way of optimizing the system as efficiently and precisely as possible, as well as minimizing time and material wastage. An experimental design was therefore used to develop a thermoresponsive pharmaceutical composition according to the invention which is capable of providing release for at least one month. Folic acid was used as a model drug for prototyping as it had a similar solubility to methotrexate, which at the time of performing the research was too costly for prototyping studies. Nevertheless, the said pharmaceutical dosage form is not drug dependent. The effects of the concentration of PMVE and a salt, calcium chloride, on the gelation temperature, mechanical properties and drug release were investigated.

The results indicate that the addition of the calcium chloride causes the PMVE composition to form a gel at lower temperatures. In addition, the release of folic acid from these implants was slow and continued for longer than a month. The slow release of folic acid from the implant could have potential therapeutic benefits, as not only is the release prolonged but also the amount being released at the tumour site will be controlled, and as a result the damage to the surrounding healthy tissue will be less compared to a formulation which releases the drug too quickly—this could cause a very high dose at the site, leading to tissue damage. These drug release results therefore appear promising for the use of an implant-forming system for the prolonged delivery of drug in the treatment of solid tumours.

The microparticle formulations were shown to have quick release, which will offset the prolonged release from the implant. This will result in higher amounts of drug in the tumor area.

The micro- or nano-particles can release the active agents or drugs at the site of the tumour for prolonged periods of time and can release the drugs faster on reaching physiological pH. Drug release was conducted at 3 pH values as follows: 5.6, 6.75 and 7.4. Drug is released faster at the lower pH of 5.6, slower at 6.75 and rapidly at 7.4. Due to the enhanced permeation and retention effect (Maeda et al, 2000), most of the particles will remain at the site of the tumour following diffusion out of the implanted device. Hence very few particles will reach physiological pH (pH7.4), but the few particles that do reach the bloodstream could possibly decrease potential metastasis.

The invention will now be described in more detail by way of the following non-limiting examples.

### EXAMPLES

### Materials

### Thermoresponsive Polymer Solutions

Poly(methyl vinyl ether) (PMVE) (50% wt in water), folic acid and dialysis tubing (MWCO 12400 kDa, flat width 32 mm) were purchased from Sigma-Aldrich (Steinheim, Germany). Calcium chloride was purchased from Rochelle Chemicals (Johannesburg, South Africa). All other substances were of analytical grade and all solutions were prepared using Milli-Q grade water.

### Microparticles

Chitosan (medium molecular weight) (CHT), acetic acid, sodium hydroxide and folic acid were purchased from Sigma Aldrich. Poly(methacrylic acid-co-methyl methacrylate) (PMMA) Eudragit S100® was purchased from Rohm, Germany. All other chemicals were of reagent grade and were used without further purification.

### Design and Preparation of Thermoresponsive Polymer Compositions

A two-factor face-centred experimental formulation design was utilised to prepare 15 formulations containing varying amounts of polymer and salt as shown in Table 1. A 30% PMVE formulation containing 0.1M CaCl₂: 15g of PMVE (50% w/v) (p=1.03 g/mL) was diluted with 9.71 mL deionised Milli-Q water to give a 30% w/v solution. 0.142 g of CaCl₂ was then added to 10 mL of the 30% w/v PMVE solution. The solution was then stirred until a homogenous solution formed. For drug release, folic acid was added to the solutions in a concentration of 15 mg/3 mL of formulation.

TABLE 1
Composition of PMVE gel formulations

| Formulation | Concentration of PMVE (%) | Concentration of CaCl₂ (M) |
|---|---|---|
| 1 | 30 | 0.200 |
| 2 | 10 | 0.200 |
| 3 | 20 | 0.125 |
| 4 | 20 | 0.125 |
| 5 | 10 | 0.050 |
| 6 | 30 | 0.050 |
| 7 | 20 | 0.125 |
| 8 | 20 | 0.200 |
| 9 | 30 | 0.125 |
| 10 | 20 | 0.125 |
| 11 | 20 | 0.125 |
| 12 | 20 | 0.125 |
| 13 | 10 | 0.125 |
| 14 | 20 | 0.050 |

### Determination of the Gelation Temperature and Determination of the Viscoelastic Behaviour of the Thermoresponsive Polymer Compositions

A Haake Modular Advanced Rheometer System (ThermoFisher Scientific, Germany) fitted with a 2° Titanium probe was used for these studies. Stress sweeps at 0.1 Hz, 1 Hz and 10 Hz were conducted on the samples to determine the linear viscoelastic region for the formulations. Using the information obtained, samples were then exposed to a fixed strain and oscillation (18 Pa, 10 Hz) while the temperature of the sample was increased from 20-40° C. in a ramped temperature flow curve (0.33°/min). The lower critical solution temperature (LCST) was defined as the temperature at which there was a significant increase in the storage modulus (G′) and dynamic viscosity (η′). All tests were conducted in duplicate. The dynamic viscosity (η′) of each of the formulations at 37.5° C. was also determined.

Determination of the Release of Folic Acid from the Implant Under Conditions Mimicking those at the Tumour Site

Drug release studies were conducted in an orbital shaker bath (37.5° C., 25 rpm). A dialysis tubing method similar to that described by Graves et al, 2007 was used. The dialysis tubing (MWCO: 12000 kDa) was thoroughly rinsed to remove preservative fluid and was then cut into pieces measuring 8.5 cm. One end of the tubing was tied and the tubing was filled with 8 mL of dissolution fluid (phosphate buffered saline (PBS), pH 6.75). 3 mL of the formulation were injected into the dialysis tubing and the other end of the tubing was also tied. The dialysis bags were then placed into jars filled with 100 mL of phosphate buffered saline (pH 6.75) and the jars were placed into a shaker bath. 10 mL samples were drawn at the following intervals: 6 hours, 1 day, 3 days, 5 days, 9 days, 13 days, 17 days, 22 days, 27 days, 31 days and 40 days. 10 mL of pre-warmed buffer were replaced at each time interval to maintain sink conditions. Samples were analysed using a UV spectrophotometer at the wavelength for folic acid (280 nm).

Determination of the Force of Injectability: The force Required to Inject the Implant at the Tumor Site

In order to develop a formulation which will be clinically beneficial, the formulations must be easily injectable. PMVE, as supplied (50% w/v solution), is highly viscous and for this reason the injectability of the formulations was tested. A Textural Analyser (TA.XTplus Texture Analyser, StableMicroSystems, England) was fitted with a 2 mm cylindrical steel probe and a 5 kg load cell and the samples were tested for the ability to be easily injected. The maximum force required to depress the plunger of a syringe filled with the implant formulation was determined and compared with the force required to depress the plunger of a water filled syringe. A typical test involved advancing the probe at a predetermined velocity into the sample in accordance with the following parameters: pre-test and post-test speeds 1 mm/s and 3mm/s respectively; test speed 2 mm/s; maximum compression force 40 N; trigger force 0.001 N. Data acquisition was performed at 200 points/sec via Texture Exponent for Windows software, Version 3.2.

### Methods of Preparation of Microparticles

Formulations of microparticles were prepared as summarized in Table 2 and in each formulation the concentration of polymers (0.1-0.5%) or the sonication time (5 min-40 min) was altered. To produce formulation 1, a solution of medium molecular weight chitosan (CHT) (0.5% w/v) was prepared by adding 0.5 g of chitosan to 100 mL of a 2% acetic acid solution. 0.05 mL of Span 80 was added to 10 mL of the CHT solution. A solution of PMMA (0.5% w/v) was then prepared by dissolving 0.5 g of a 1M NaOH solution and 30 mg of folic acid was added to this solution. 10 mL of the CHT solution was placed under the sonicator (Vibra-Cell Ultrasonicator, Sonics, USA) and 10 mL of the second solution (PMMA and folic acid) was added to the solution being sonicated. After the formulations were prepared, they were left for 24 hours to cure. The particles settled to the bottom and the supernatant was discarded. The particles were collected and frozen for 24 hours. Formulations were then lyophilised for 48 hours.

TABLE 2
Design of particle formulations

| Formulation | CHT/PMMA (% w/v) | Time (min) |
|---|---|---|
| 1 | 0.5 | 40 |
| 2 | 0.1 | 40 |
| 3 | 0.3 | 22.5 |
| 4 | 0.3 | 22.5 |
| 5 | 0.1 | 5 |
| 6 | 0.5 | 5 |
| 7 | 0.3 | 22.5 |
| 8 | 0.3 | 40 |
| 9 | 0.5 | 22.5 |
| 10 | 0.3 | 22.5 |
| 11 | 0.3 | 22.5 |
| 12 | 0.3 | 22.5 |
| 13 | 0.1 | 22.5 |
| 14 | 0.3 | 5 |

### Determination of Drug Entrapment Percentages and Particle Yield

To determine the amount of drug entrapped within the microparticles, accurately weighed amounts of each sample (approximately 25 mg) were placed in 10 mL of 0.01M NaOH. These samples were left for 48 hours to ensure complete breakdown of the particles. Following centrifugation, supernatant was assayed and drug concentration was determined. The following equation was used to determine drug entrapment:

$$Entrapment Efficacy = \frac{A}{B} \times 100 \qquad Equation 1$$

where A is the drug concentration in the micro- or nano-particles (mg/mL) and B is the theoretical drug concentration (mg/mL).

### Examination of the Morphology of the Particles

In order to assess the size and shape of the prepared formulations, electron microscopy was undertaken using a scanning electron microscope, Phenom (FEI, USA). Samples were mounted onto stubs using carbon tape and sputter-coated with gold under an argon atmosphere using a sputter-coater (SPI, USA) for 120 seconds.

Determination of the Release of Folic Acid from the Implant When Cooled by the Application of Ice Under Conditions Mimicking those at the Tumour Site.

Drug release studies were conducted in an orbital shaker bath (37.5° C., 25 rpm). A dialysis tubing method similar to that described by Graves et al, 2007 was used. The dialysis tubing (MWCO: 12000 kDa) was thoroughly rinsed to remove preservative fluid and was then cut into pieces measuring 8.5 cm. One end of the tubing was tied and the tubing was filled with 8 mL of dissolution fluid (phosphate buffered saline (PBS), pH 6.75). 3 mL of the formulation were injected into the dialysis tubing and the other end of the tubing was also tied. The dialysis bags were then placed into vessels filled with 100 mL of phosphate buffered saline (pH 6.75) and the vessels were placed into a shaker bath. 10 mL samples were drawn at the following intervals: 1 hour, 2 hours and 3 hours. Following each withdrawal of sample, the entire vessel was placed in an ice bath for 5 mins and a sample was then drawn. 10 mL of pre-warmed buffer were replaced at each time interval to maintain sink conditions. Samples were analysed using a UV spectrophotometer at the wavelength for folic acid (280 nm).

### Results and Discussion

Assessment of the Rheological Properties of the in situ Forming Implant

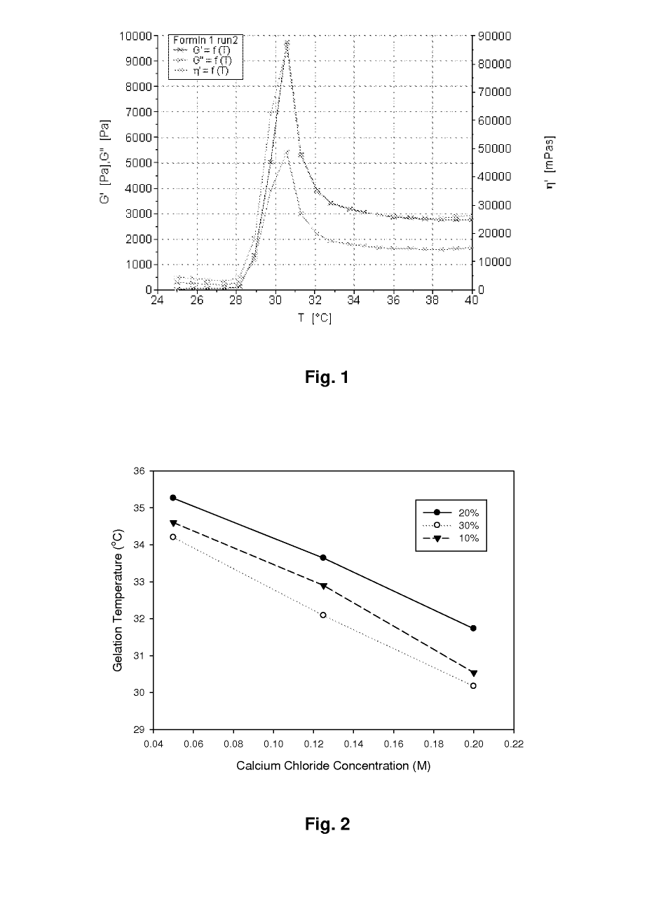

Formulations all showed a gelation temperature of less than 36° C. (FIG. 1). As shown in FIG. 2, the gelation temperature of PMVE-CaCl₂ formulations depends on the concentration of calcium chloride in the formulation. Higher amounts of calcium chloride result in formulations which gel at lower temperatures. Interestingly, the formulations with a 20% w/v concentration of PMVE have a higher gelation temperature than would be expected from the results for PMVE 10% w/v and 30% w/v.

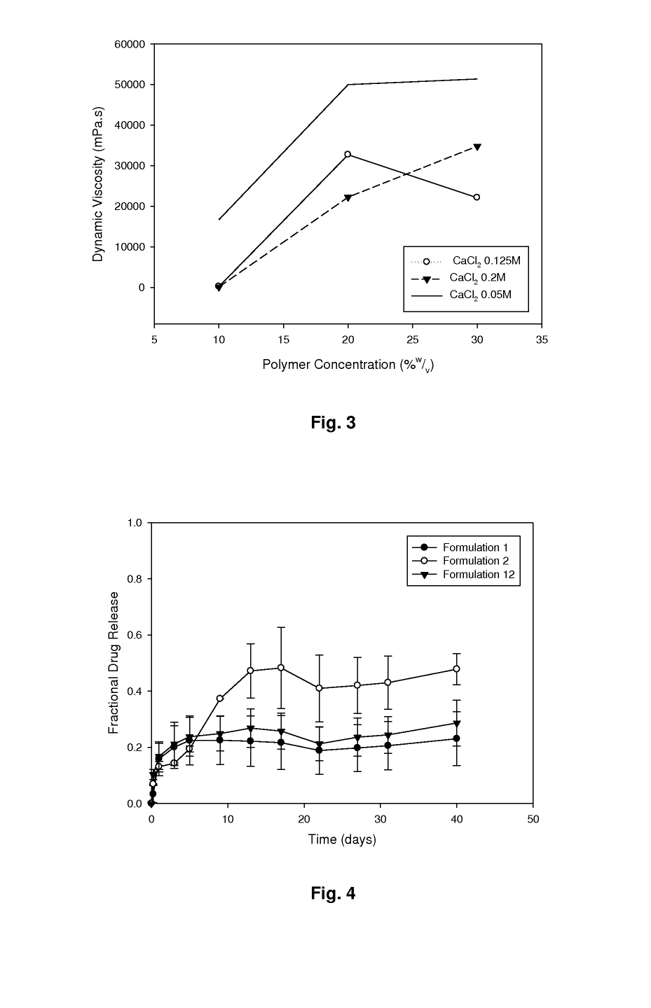

The dynamic viscosity and the concentration of the polymer, PMVE, are almost linearly related in the case of formulations containing 0.2M calcium chloride (FIG. 3). With an increase in polymer concentration, there is a corresponding increase in the dynamic viscosity. This effect is lesser in the formulations with higher or lower amounts of calcium chloride in formulations containing 30% PMVE. Conversely, there is a decrease in the dynamic viscosity with an increase in salt concentration. This is due to the gelation of the formulations containing higher amounts of calcium chloride at lower temperatures, whereas the formulations with low amounts of the salt have only begun to gel at temperatures around 37° C. As the implant forms at a lower temperature, the burst release of the drug is reduced.

Release of Folic Acid from the in situ Forming Implants

The release of drug from all the formulations exceeded a month (FIG. 4). The release appears dependant on the polymer concentration in the formulation, as the release is slowest from formulation 1 which is composed of 30% w/v PMVE and fastest from the formulation containing 10% w/v PMVE (formulation 2). The effect of increasing concentrations of calcium chloride on the release profiles is also interesting. For formulations containing 10% PMVE, the higher the concentration of calcium chloride, the slower the release of folic acid from the formulation. However, the same effect is not as pronounced with formulations having a higher concentration of PMVE 20% w/v and the effect is reversed in the case of 30% w/v PMVE.

### Force Required to Insect the Implants

Implants containing higher amounts of polymer required a greater force to be injected and the addition of the salt, calcium chloride, had no effect on the force required to inject the implants.

### Entrapment of Drug Within the Micro-Particles

Most of the formulations showed fairly good entrapment ranging from 55 to 74%. The entrapment did not appear to be dependent on either the sonication time or the concentration of the polymers.

Release of Folic Acid from the Micro-Particles

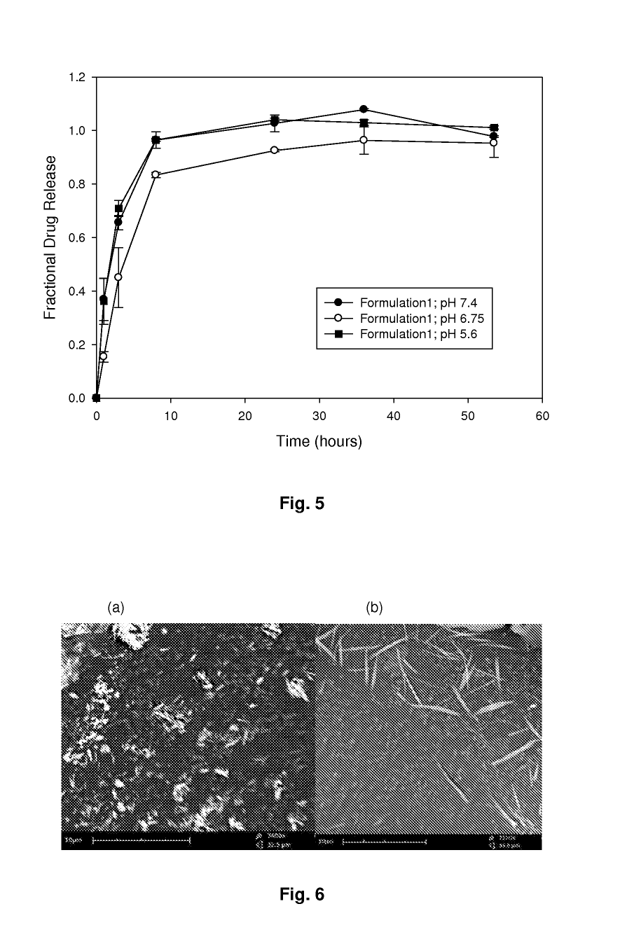

As shown in FIG. 5, release of folic acid from the particles was conducted in two buffered saline solutions, pH 6.75 and pH 7.4, and showed slightly different profiles. Release of the drug from the particles appears to be pH responsive with the release of folic acid faster at pH 6.75, where the release was complete after 36 hours. In buffered saline at pH 7.4, the release from the particles lasted 54 hours. This can be attributed to the swelling of polymers at the higher pH, resulting in slower diffusion of the drug from the particles. As mentioned previously, the pH of a tumour is lower than that of physiological pH. When the particles diffuse out of the implant, the pH of the tumour will be approximately 6.75. Although release of the drug is faster at a pH value of 7.4 as can be seen in FIG. 5, very little drug is expected to reach the blood stream due to the enhanced permeation and retention effect. Should the tumour have a lower pH as is associated with rapidly growing tumours, the release will be faster as seen with the release at pH 5.6. (FIG. 5). Since intracellular pH is about 5.6, the similar principle will be of importance if the particles were able to move into the cells. However, this is unlikely considering the current size of the particles.

### Determination of the Morphology of the Particles Using SEM

As can be seen from the electron micrographs in FIG. 6, the particles had peculiar spindle-like morphology and single spindles can be seen in FIG. 6b and clumps in FIG. 6a. The size of the spindles ranged from 1.64 μm-9.883 μm in length and widths ranged from 0.29-0.6 μm.

### Optimisation of the Implant and the Microparticles

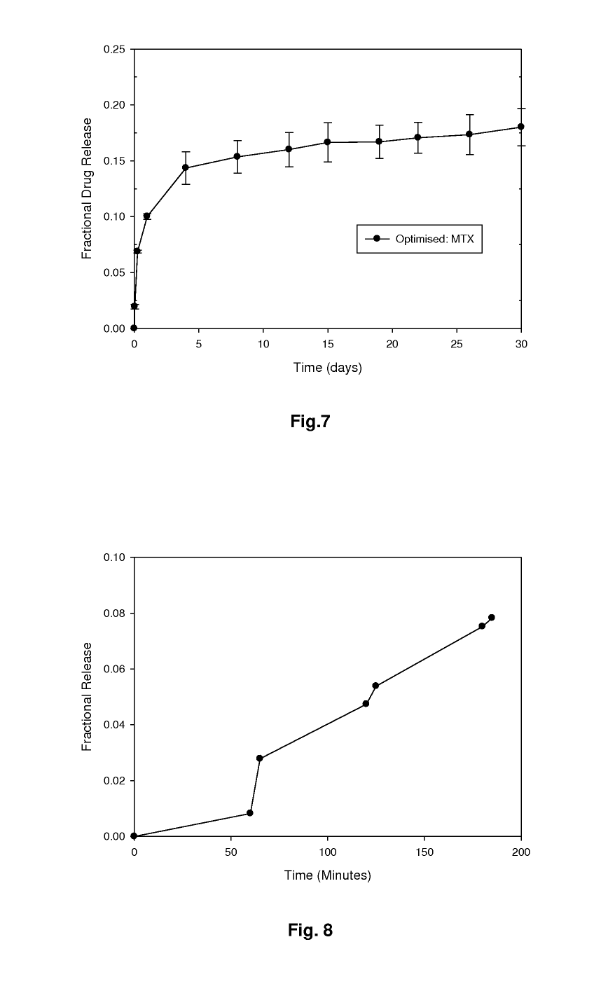

Using the mean dissolution time, the force of injectability and the gelation temperature as responses, the implant was optimized using Minitab V15 (Minitab® Inc, PA, USA). The same program was used to optimise the particles and here the release of the drug at two pHs and drug entrapment efficacy was used as the responses. The obtained optimised implant (16.87% PMVE and 0.1482M calcium chloride) was then subjected to the 3 responses and FIG. 7 shows release from the implant when loaded with methotrexate. The optimised implant also showed an optimum injectability and had a thermal gelation temperature of 32.46° C. Release from the optimised particles is as shown in FIG. 5.

Determination of the Release of Folic Acid from the Implant Under Conditions Mimicking those at the Tumour Site.

As shown in FIG. 8, the release of the drug from the implant can easily be altered with the application of ice. The steps indicate the application of ice and the corresponding increase in release of drug from the implant. For example, a patient who had an implant with, for example, a pain killer, could rapidly release the pain killer by applying an ice pack onto the area of the skin in the region of the implant.

### REFERENCES

Argyriou A A, Antonacopoulou A, lconomou G, and Kalofonos H. P.2009. Treatment options for malignant gliomas, emphasizing towards new molecularly targeted therapies. Critical Reviews in Oncology/Hematology, 69:99-210.

Brem H, Piantadosi S, Burger P C, Walker M, Selker R, Vick N A, Black K, Sisti, M, Brem S, Mohr G, Muller P, Morawetz R, Schold S C. 1995. Placebo controlled trail of safety and efficacy of intraoperative controlled delivery by biodegradable polymers of chemotherapy for recurrent gliomas. The Lancet, 345: 1008-1012.

Chopko B. W., (2009). Intracavitary carmustine polymer implantation as an adjunct to palliative metastatic vertebral column tumors Surgical Neurology. Article in Press. doi:10.1016/j.surneu.2008.12.015.

Felice P, Corinaldesi G, Lizio G. 2009. Implant Prosthetic Rehabilitation of Posterior Mandible After Tumor Ablation With Inferior Alveolar Nerve Mobilization and Inlay Bone Grafting: A Case Report. Journal of Oral and Maxillofacial Surgery. 67:1104-1112.

Graves R, Freeman T and Mandal T K 2007 In vitro dissolution method for evaluation of buprenorphine in situ gel formulation: A technical note. AAPA Pharmaceutical Science and Technology. 8(3):

Hatefi A and Amsden B, 2002. Biodegradable injectable in situ forming drug delivery systems. Journal of controlled release, 80: 9-28

Hong L T, Joon P and Whan L J. 2007. In situ controlled release drug delivery system. EP Patent KR 20070100701

Karayanni K and Staikos G. 2000. Study of the lower critical solution temperature behaviour of poly(vinyl methyl ether) aqueous solutions in the presence of poly(acrylic acid). The role of interpolymer hydrogen bonding interaction. European Polymer Journal, 36: 2645-2650

Klouda L and Mikos A G. 2008. Review article: Thermoresponsive hydrogels in biomedical applications. European Journal of Pharmaceutics and Biopharmaceutics, 68:34-45.

Lewis G A, Mathieu D and Phan-Tan-Luu R. 1999. Pharmaceutical Experimental Design, Marcel Dekker Inc.: New York: USA

Madbouly S A and Ougizawa T, 2005. Thermal Croslinking of Poly(Vinyl Methyl Ether). I. Effect of Crosslinking Process on the viscoelastic properties. Journal of Macromolecular Science, 43(2): 471-487

Maeda H, Wu J, Sawa T, Matsumura Y and Hori K. 2000. Tumour vascular permeability and the EPR effect in macromolecular therapeutics: a review, Journal of Controlled Release, 65: 271-284

Marceij R, Youqing S, Huadong T. 2007. Nanoparticles for cytoplasmic delivery of drugs to cancer cells. EP Patent 2007001356.

Martin G and Jain R K. 1994. Non-invasive measurement of interstitial pH profiles in normal and neoplastic tissue using fluorescence ratio imaging microscopy. Cancer Research, 54:5670-4.

Nurkeeva Z S, Tyukova I S, Suvorova A I, Mun G A, Dzhusupbekova A B, Khutoryanskiy V V. 2005. Miscibility studies in poly(methyl vinyl ether)/hydroxypropylcellulose binary system in aqueous solutions and solid state. Carbohydrate Polymers. 62:80-86

Packhaeuser C B, Schnieders J, Oster C G, Kissel T. 2004. In situ forming parenteral drug delivery systems: an overview. European Journal of Pharmaceutics and Biopharmaceutics, 58:445-455

Perry J, Chambers A, Spithoff K, and Laperriere N. 2007. Gliadel wafers in the treatment of malignant glioma: a systematic review. Current Oncology. 14(5): 189-194.

Richter S, 2004. Mechanical properties of poly(vinyl methyl ether) hydrogels below and above their volume phase transition. Colloid Polymer Science, 282: 1213-1221

Sinha. Chitosan microspheres as potential carriers for drugs. International Journal of Pharmaceutics, 274: 1-33

Urtti, A, 1985. Pilocarpine release from matrices of alkyl half esters of poly(vinyl methyl ether/maleic anhydride). International Journal of Pharmaceutics 26:45-55

Woolfson A D, McCafferty D F and Moss G P. 1998. Development and Characterisation of a moisture-activated bioadhesive drug delivery system for the percutaneous local anaesthesia. International Journal of Pharmaceutics, 169: 83-949.

Yahara T, Koga T, Yoshida S, Nakagawa S, Deguchi H and Shirouzu K. 2003. Relationship between microvessel density and thermographic hot areas in breast cancer. Surgery Today. 33: 243-248.

1. A pharmaceutical composition for the delivery of a pharmaceutically active agent, the composition comprising:
a thermoresponsive polymer composition which is in a liquid form at or about room temperature and in a solid or gelatinous form at or about body temperature, wherein the thermoresponsive polymer composition is formed from cross-linked poly(methyl vinyl ether) (PMVE) and an inorganic salt; and
a plurality of micro- or nano-particles which are pH responsive and which include at least one pharmaceutically active agent;
wherein the micro- or nano-particles are suspended in the thermoresponsive polymer composition.

2. The pharmaceutical composition according to claim 1, wherein the inorganic salt is calcium chloride or sodium hydrogen phosphate.

3. The pharmaceutical composition according to claim 2, wherein the thermoresponsive polymer composition comprises a second polymer selected from the group consisting of gum Arabic, carageenan, hydroxypropyl cellulose (HPC), methylcellulose (MC), ethylcellulose and hydroxypropylmethylcellulose (HPMC).

4. The pharmaceutical composition according to claim 1, wherein the thermoresponsive polymer composition comprises PMVE, a cellulosic polymer and an inorganic salt.

5. The pharmaceutical composition according to claim 4, wherein the cellulosic polymer is methylcellulose or ethylcellulose.

6. The pharmaceutical composition according to claim 1, wherein the micro- or nano-particles comprise at least two pH responsive polymers.

7. The pharmaceutical composition according to claim 1, wherein the micro- or nano-particles are insoluble at the pH of healthy tissue but soluble at the pH of cancer cells.

8. The pharmaceutical composition according to claim 7, wherein the pH-reponsive polymers are chitosan and eudragit.

9. The pharmaceutical composition according to claim 1, wherein the micro- or nano-particles comprise additional polymers to stabilise the particles and/or to enhance entrapment of the pharmaceutically active agent.

10. The pharmaceutical composition according to claim 9, wherein the additional polymers are alginates and/or HPMC.

11. The pharmaceutical composition according to claim 1, wherein the pharmaceutically active agent is a chemotherapeutic agent.

12. The pharmaceutical composition according to claim 11, wherein the chemotherapeutic agent is for treating a solid tumour.

13. The pharmaceutical composition according to claim 1, wherein the pharmaceutically active agent is for treating pain.

14. The pharmaceutical composition according to claim 1, wherein the pharmaceutically active agent is released in a sustained and controlled manner for a long-term therapeutic effect.

15. The pharmaceutical composition according to claim 1, wherein the thermoresponsive polymer composition comprises at least a second pharmaceutically active agent.

16. The pharmaceutical composition according to claim 1, which is an injectable formulation.

17. A method of introducing a pharmaceutically active agent to a specific site within a human or animal comprising introducing to the specific site in the human or animal a pharmaceutical composition of claim 1.

18. A method of treating a solid cancerous tumour comprising injecting a pharmaceutical composition according to claim 1 percutaneously to the site of the tumour.

19. A method of formulating a pharmaceutical composition according to claim 1, the method comprising the steps of:
forming pH responsive micro- or nano-particles from at least two cross-linked pH responsive polymers and at least one pharmaceutically active agent;
forming a thermoresponsive polymer composition from cross-linked poly(methyl vinyl ether) (PMVE) and an inorganic salt; and
mixing the micro- or nano-particles with the thermoresponsive polymer composition so that the micro- or nano-particles are suspended in the theremoresponsive polymer composition.

20. A method according to claim 19, wherein the inorganic salt is calcium chloride.
